(19) United States
(12) Patent Application Publication
(10) Pub. No.: US 2026/0345035 A1
(43) Pub. Date: Nov. 5, 2026

(54) BLOOD GLUCOSE MONITORING MODULE WITH COMPOSITE-WAVELENGTH LIGHT SOURCE AND WEARABLE DEVICE COMPRISING THE SAME

(71) Applicant: Taiwan-Asia Semiconductor Corporation, Hsinchu City (TW)

(72) Inventor: Ching-Pu Tai, Hsinchu City (TW)

(21) Appl. No.: 19/655,187

(22) Filed: Apr. 22, 2026

(30) Foreign Application Priority Data
Apr. 30, 2025 (TW) 114116373

(51) Int. Cl. A61B 5/145; A61B 5/00; A61B 5/1455

(52) U.S. Cl. CPC A61B 5/14532; A61B 5/1455; A61B 5/7235; A61B 2560/0214; A61B 2562/166

(57) ABSTRACT

A blood glucose monitoring module with composite-wavelength light source and a wearable device comprising the same. The blood glucose monitoring module comprises a light-emitting component and a light-sensing component. The light-emitting component includes a plurality of light source elements configured to emit a plurality of light beams toward a subject. The light beams have wavelengths ranging from 700 nm to 1700 nm. The light beams include at least one first wavelength band and at least one second wavelength band. The at least one first wavelength band is for calibrating depth-dependent optical properties of skin tissue. The at least one second wavelength band is for detecting absorption intensity associated with blood glucose. The light-sensing component is disposed adjacent to the light-emitting component and is configured to receive diffusely reflected optical signals reflected from the subject after being illuminated by the light beams, thereby acquiring optical characteristics indicative of blood glucose concentration.

### CROSS-REFERENCES TO RELATED APPLICATIONS

This application claims the benefit of priority to Taiwanese Patent Application No. 114116373 filed on Apr. 30, 2025, which is hereby incorporated by reference in its entirety.

### BACKGROUND OF THE INVENTION

### Field of the Invention

This invention relates to a blood glucose monitoring module having a composite-wavelength light source and to a wearable device comprising the same, and in particular to a blood glucose monitoring module that employs a light-emitting diode light source capable of simultaneously providing composite-wavelength light in a wavelength range from near-infrared to short-wave infrared, and that performs non-invasive blood glucose concentration detection by diffuse reflectance infrared spectroscopy.

### Descriptions of the Related Art

In recent years, to reduce discomfort associated with invasive blood glucose measurements, various non-invasive blood glucose detection techniques have been developed. Among known non-invasive blood glucose monitoring technologies, common approaches utilize the spectral absorption characteristics of near-infrared (NIR) light or short-wave infrared (SWIR) light to estimate blood glucose concentration by detecting the selective absorption of glucose molecules at specific wavelengths within biological tissue. In some techniques, a single-wavelength laser light source is used as an excitation source. Raman photons generated by interactions between the laser light and glucose molecules located at different depths within skin tissue are collected, and Raman scattering signals with different energy levels are analyzed by comparing Raman spectra to calculate blood glucose concentration. In addition, some studies combine spectral imaging systems, transmissive laser optical paths, and calibration algorithms to improve detection accuracy and computational precision.

However, the above-described detection systems still suffer from several drawbacks. First, energy focusing of a single-wavelength laser light source typically requires complex optical component designs, such as lenses, mirrors, and dichroic mirrors, in order to compensate for energy losses caused by depth differences in biological tissues. Such configurations increase system size and cost, and are not suitable for miniaturized and wearable applications. Second, Raman signals are inherently weak and therefore require high-power laser excitation and highly sensitive spectrometers, which results in high power consumption and potential overheating of the detection device. Furthermore, most existing technologies are unable to simultaneously capture glucose-related signal features at different tissue depths, causing the detection results to be susceptible to environmental variations or individual differences. Accordingly, the accuracy and stability of such systems still require further improvement.

In view of the above, the present invention provides a blood glucose monitoring module having a composite-wavelength light source and a wearable device comprising the same, so as to overcome the problems of the prior art, including the need for lens-based optical focusing to compensate for energy loss when using a single-wavelength laser light source, insufficient sensing accuracy, and structural complexity that hinders miniaturization.

### SUMMARY OF THE INVENTION

The objective of the invention is to provide a novel blood glucose monitoring module having a composite-wavelength light source and a wearable device comprising the same. The blood glucose monitoring module of the present invention employs a composite-wavelength light-emitting diode light source capable of simultaneously covering wavelength bands from near-infrared (NIR) to short-wave infrared (SWIR), for example, within a wavelength range from about 700 nanometers to about 1700 nanometers, to replace a conventional single-wavelength laser light source. By utilizing differences in tissue penetration capability among different wavelength bands, layered detection of glucose signals at different depths within biological tissue can be achieved. Moreover, the higher energy characteristics of shorter-wavelength light can compensate for the lower energy characteristics of longer-wavelength light, thereby increasing wavelength feature information and improving the accuracy of blood glucose concentration calibration. In addition, because the LED light source provides a wide-angle emission characteristic, the present invention can realize a diffuse reflectance optical effect without requiring focusing optical components or optical fiber transmission paths that are necessary in Raman spectroscopy-based techniques. As a result, the optical system structure can be simplified, device size can be effectively reduced, and a miniaturized wearable device suitable for non-invasive blood glucose detection can be achieved. Overall, the blood glucose monitoring module provided by the present invention enables a blood glucose detection application that combines non-invasiveness, high sensitivity, high accuracy, and miniaturization.

To achieve the above objective, this invention provides a blood glucose monitoring module with a composite-wavelength light source comprising a light-emitting component and a light-sensing component. The light-emitting component includes a plurality of light source elements configured to emit a plurality of light beams toward a subject. The light beams have wavelengths in a range from about 700 nanometers (nm) to about 1700 nanometers (nm). The light beams include at least one first wavelength band and at least one second wavelength band. The at least one first wavelength band is configured to calibrate depth-dependent optical properties of skin tissue. The at least one second wavelength band is configured to detect absorption intensity associated with blood glucose. The light-sensing component is disposed adjacent to the light-emitting component and configured to receive a plurality of diffusely reflected optical signals reflected from the subject after being illuminated by the light beams, so as to acquire optical characteristics indicative of a blood glucose concentration of the subject.

In one embodiment of the blood glucose monitoring module of this invention, the at least one first wavelength band has a wavelength range from about 700 nanometers to about 1100 nanometers.

In one embodiment of the blood glucose monitoring module of this invention, the at least one second wavelength band has a wavelength range from about 1100 nanometers to about 1700 nanometers.

In one embodiment of the blood glucose monitoring module of this invention, the blood glucose monitoring module further comprises a substrate, and the light-emitting component and the light-sensing component are disposed on the same side of the substrate.

In one embodiment of the blood glucose monitoring module of this invention, the light-emitting component and the light-sensing component are disposed corresponding to each other, the light-emitting component is disposed at one end of the same side of the substrate, and the light-sensing component is disposed at the other end of the same side of the substrate.

In one embodiment of the blood glucose monitoring module of this invention, the light source elements comprise a plurality of light-emitting diodes.

In one embodiment of the blood glucose monitoring module of this invention, the light-sensing component comprises a plurality of photodiodes.

In another embodiment of the blood glucose monitoring module of this invention, the blood glucose monitoring module further comprises an optical filter configured to selectively pass the at least one first wavelength band or the at least one second wavelength band.

In one embodiment of the blood glucose monitoring module of this invention, the light-sensing component further comprises a data processing unit configured to receive and analyze spectral signals of the diffusely reflected optical signals, and to output data corresponding to the blood glucose concentration by comparing the spectral signals with reference spectra.

To achieve the above objective, this invention further provides a wearable device, which comprises the aforementioned blood glucose monitoring module with a composite-wavelength light source, a housing, a power element, and at least one printed circuit board assembly. The housing is configured to accommodate the blood glucose monitoring module. The power element is disposed within the housing. The power element is coupled to the blood glucose monitoring module, and configured to supply power. The at least one printed circuit board assembly is disposed within the housing. The at least one printed circuit board assembly is coupled to the blood glucose monitoring module, and configured to control operation of the blood glucose monitoring module.

After referring to the drawings and the embodiments as described in the following, those the ordinary skilled in this art can understand other objectives of the present invention, as well as the technical means and embodiments of the present invention.

### BRIEF DESCRIPTION OF THE DRAWINGS

FIG. 1 is a schematic view of a blood glucose monitoring module with a composite-wavelength light source according to the present invention;

FIG. 2 is a schematic view of light source elements of the blood glucose monitoring module with a composite-wavelength light source according to the present invention; and

FIG. 3 is a cross-sectional schematic view of a wearable device according to the present invention.

### DETAILED DESCRIPTION OF THE PREFERRED EMBODIMENT

In the following description, the present invention will be explained with reference to various embodiments thereof. These embodiments of the present invention are not intended to limit the present invention to any specific environment, application or particular method for implementations described in these embodiments. Therefore, the description of these embodiments is for illustrative purposes only and is not intended to limit the present invention. It shall be appreciated that, in the following embodiments and the attached drawings, a part of elements not directly related to the present invention may be omitted from the illustration, and dimensional proportions among individual elements and the numbers of each element in the accompanying drawings are provided only for ease of understanding but not to limit the present invention.

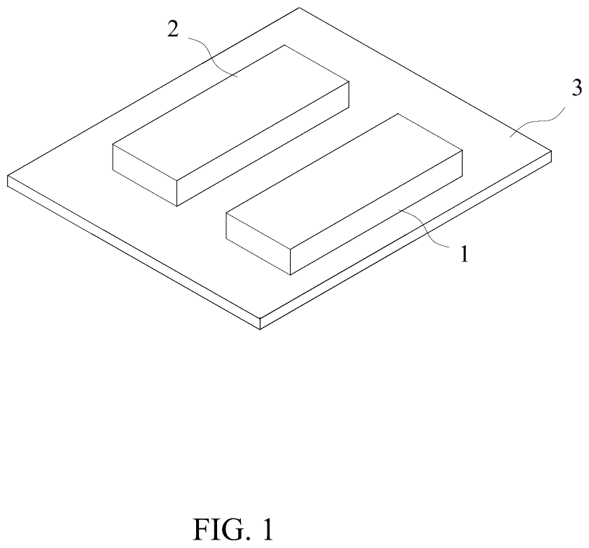

Please refer to FIG. 1, a blood glucose monitoring module with a composite-wavelength light source 100 (hereinafter referred to as the “blood glucose monitoring module 100”) according to one embodiment of the present invention comprises a light-emitting component 1, a light-sensing component 2, and a substrate 3. The substrate 3 is disposed at a lowermost layer of the blood glucose monitoring module 100 and serves as a supporting structure for the components. The light-emitting component 1 and the light-sensing component 2 are disposed on an upper surface of the substrate 3. In addition, the blood glucose monitoring module 100 may further comprise an optical filter 4 disposed above the light-emitting component 1 and the light-sensing component 2. The specific position of the optical filter 4 may be adjusted according to actual detection requirements and is not limited herein. The primary purpose of the blood glucose monitoring module 100 is to emit composite light beams having a plurality of different wavelengths toward a subject through the light-emitting component 1. When the light beams irradiate glucose molecules within the subject and generate a plurality of diffusely reflected optical signals, the light-sensing component 2 receives the diffusely reflected optical signals so as to obtain optical characteristics indicative of a blood glucose concentration of the subject.

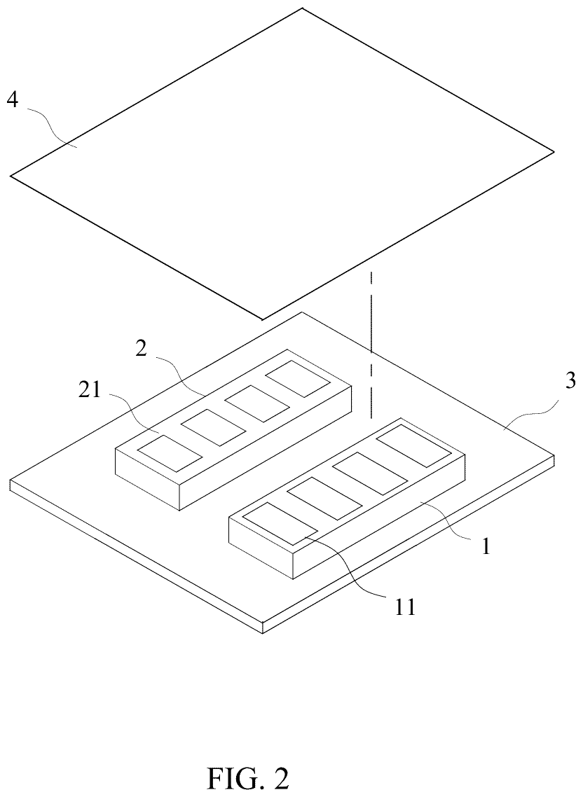

Referring to FIG. 2, the light-emitting component 1 comprises a plurality of light source elements 11. The light source elements 11 serve as emission sources of the light-emitting component 1 and are configured to emit a plurality of light beams having wavelengths in a range from about 700 nanometers to about 1700 nanometers toward the subject. The light beams include at least one first wavelength band and at least one second wavelength band. In one embodiment, the first wavelength band has a wavelength range from about 700 nanometers to about 1100 nanometers, which falls within a NIR range and is configured to calibrate depth-dependent optical properties of skin tissue, so as to compensate for scattering and absorption effects occurring at different tissue depths, such as within an epidermis layer, a dermis layer, and a stratum corneum. On the other hand, the second wavelength band has a wavelength range from about 1100 nanometers to about 1700 nanometers, which falls within a SWIR range and is configured to detect absorption peaks of glucose molecules within this wavelength range, thereby obtaining corresponding absorption spectral signals. In a preferred embodiment, the light source elements 11 are LED light sources and may be implemented in the form of LED chips configured for different wavelengths to provide composite-wavelength light beams. Materials of the LEDs may include gallium arsenide (GaAs), indium phosphide (InP), or indium gallium arsenide (InGaAs), and may be flexibly selected according to target wavelengths to be detected, without limitation. For example, in one embodiment of the present invention, the blood glucose monitoring module 100 comprises four light source elements 11 configured to emit one first wavelength band and three second wavelength bands. The first wavelength band has a shorter wavelength and higher energy, providing enhanced tissue penetration capability to penetrate into deeper tissue regions, such as below a dermis layer, and to obtain the diffusely reflected optical signals generated by glucose molecules at deeper depths for depth calibration. The second wavelength band has a longer wavelength and lower energy compared to the first wavelength band, but exhibits a higher absorption rate with respect to glucose molecules, thereby improving sensitivity and accuracy of blood glucose concentration detection. The composite-wavelength configuration provides excellent tissue penetration capability, enabling sensing depths reaching at least a dermis layer, and allows effective detection of glucose absorption spectral signals at different tissue depths, thereby obtaining spectral information with higher resolution and accuracy. It should be noted that the number, wavelength ranges, and combinations of the first wavelength band and the second wavelength band emitted by the light-emitting diode light sources may be adjusted according to molecular characteristics and conditions of the subject, without limitation.

The light-sensing component 2 is configured to receive a plurality of reflected optical signals that result from diffuse reflection of the light beams emitted by the light-emitting component 1 after interaction with skin tissue. The reflected optical signals primarily include the diffusely reflected optical signals originating from different depth regions within the skin tissue and contain spectral information associated with blood glucose absorption. Specifically, after the light beams are emitted from the light source elements 11 of the light-emitting component 1, the light beams selectively pass through the optical filter 4 as the first wavelength band or the second wavelength band, irradiate the skin tissue, and are then collected by the light-sensing component 2 as the diffusely reflected optical signals from the skin tissue. The light-sensing component 2 further comprises a data processing unit 21 configured to process spectral information of the received diffusely reflected optical signals and convert the spectral information into corresponding electrical signals. By comparing the electrical signals with reference spectra and performing system-based calculations, corresponding blood glucose concentration values can be obtained. In this embodiment, the light-sensing component 2 comprises a plurality of photodiodes (PDs). Each individual photodiode is configured to correspond to a specific wavelength band and to convert the diffusely reflected optical signals at the corresponding wavelength into electrical signals.

In another embodiment of the present invention, as shown in FIG. 2, the optical filter 4 may be disposed above the light-emitting component 1 and the light-sensing component 2 on the same side of the substrate 3, according to actual sensing requirements. The optical filter 4 is configured to selectively pass the first wavelength band or the second wavelength band, so as to ensure desired wavelengths of incident light and reflected optical signals and to effectively suppress stray light interference. By blocking background light having wavelengths identical to the target wavelengths but not originating from the subject, a signal-to-noise ratio (SNR) can be improved, thereby enhancing the accuracy and stability of optical analysis. In this embodiment, the optical filter 4 may be a band pass filter (BPF) configured to allow only light within a specific wavelength range to be transmitted, while filtering out light outside the target wavelength range.

The substrate 3 is configured to support the light-emitting component 1, the light-sensing component 2, and the optical filter 4, and to ensure positional stability among these components. The substrate 3 may be implemented in the form of a printed circuit board (PCB) having good thermal stability and structural strength. In this embodiment, the light-emitting component 1 and the light-sensing component 2 are disposed on the same side of the substrate 3 in a corresponding manner and are arranged with a predetermined spacing therebetween so as to improve collection efficiency of the diffusely reflected optical signals. The spacing may be adjusted according to wavelengths of the light source and actual design requirements. In particular, in the blood glucose monitoring module 100 of the present invention, the light source elements 11 of the light-emitting component 1 are disposed at one end of the same side of the substrate 3, while the light-sensing component 2 is disposed at a corresponding opposite end of the same side of the substrate 3. Such a configuration allows reduction of overall package size while maintaining sensitivity and accuracy of detection. Compared with the prior art, the present invention utilizes a wide-angle emission characteristic of light-emitting diodes to achieve diffuse reflectance without requiring a standardized optical path structure, thereby reducing use of lenses and optical components. As a result, the blood glucose monitoring module is more suitable for wearable applications and provides improved portability and practicality.

In this embodiment, a configuration and an operation process of the blood glucose monitoring module 100 are described as follows. The light-emitting component 1 comprises a plurality of light source elements 11 configured to emit a plurality of light beams including the first wavelength band and the second wavelength band toward skin tissue. In another embodiment, the light beams may be selectively filtered by the optical filter 4 to ensure that only light within a specific wavelength range is transmitted before irradiating the skin tissue. After the skin tissue is illuminated by the light beams, the diffusely reflected optical signals are generated. The diffusely reflected optical signals are then filtered by the same optical filter 4 and collected by the light-sensing component 2. The light-sensing component 2 transmits the collected diffusely reflected optical signals to the data processing unit 21 for spectral analysis and signal processing, thereby completing calculation of a blood glucose concentration.

In this embodiment, the subject is human skin tissue. The primary target molecules to be detected by the blood glucose monitoring module 100 of the present invention are glucose molecules present within the human skin tissue.

It should be noted that glucose permeates from blood vessels into interstitial fluid and is therefore present in different layers of skin tissue. To improve detection accuracy, calibration may be performed by comparing optical signals obtained from different tissue depths. As can be understood from the absorption spectra of glucose, concentration calibration can be performed based on multi-wavelength detection results, and specific wavelength bands that are sensitive to high glucose concentrations may be selected. In this embodiment, specific wavelengths within the second wavelength band exhibit significant absorption characteristics with respect to glucose and may serve as characteristic wavelengths for glucose identification. On the other hand, wavelengths that correlate with variations in glucose concentration may also be selected, and concentration determination may be performed based on scattering intensity generated thereby. Furthermore, physiological differences among individuals, such as variations in skin thickness, stratum corneum structure, race, and gender, as well as interference caused by other blood constituents such as proteins and lipids, may result in different optical responses at different wavelengths. Accordingly, relying on a single wavelength for determination may lead to misinterpretation. To reduce interference and misjudgment risk, the present invention employs a composite-wavelength light source and performs cross-comparison of multi-wavelength optical signals with infrared absorption spectra, thereby enabling more accurate determination of glucose concentration and effectively improving overall stability and reliability of analysis.

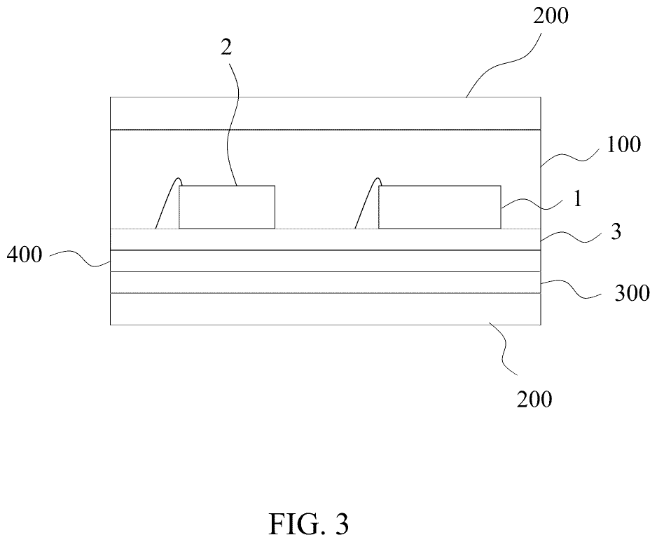

In this embodiment, the present invention further provides a wearable device 1000, as shown in FIG. 3, comprising the aforementioned blood glucose monitoring module 100 with a composite-wavelength light source, a housing 200, a power element 300, and at least one printed circuit board assembly 400. The housing 200 is configured to accommodate the blood glucose monitoring module 100, the power element 300, and the at least one printed circuit board assembly 400, so as to protect internal components and ensure stable contact between the wearable device and the subject. The power element 300 is electrically coupled to the at least one printed circuit board assembly 400 and is configured to provide power required to drive operation of the blood glucose monitoring module 100. The at least one printed circuit board assembly 400 is coupled to the blood glucose monitoring module 100 and configured to control operation processes of the blood glucose monitoring module 100, including light source driving, signal acquisition, and data processing. It should be noted that positions and arrangements of the blood glucose monitoring module 100, the housing 200, the power element 300, and the at least one printed circuit board assembly 400 may be appropriately adjusted or modified according to actual application requirements, without limitation. Through the above-described configuration, the wearable device 1000 of the present invention enables miniaturized and non-invasive blood glucose concentration detection, and provides improved portability and user convenience.

The blood glucose monitoring module 100 with a composite-wavelength light source according to the present invention employs a detection assembly capable of providing composite-wavelength illumination and thus offers technical advantages including multi-wavelength selectivity, high detection accuracy, and simplified structure. The light source elements 11 of the light-emitting component 1 are capable of simultaneously emitting multiple NIR and SWIR light beams having wavelengths in a range from about 700 nanometers to about 1700 nanometers. By utilizing composite-wavelength light beams provided by the light source elements 11, appropriate wavelengths within the first wavelength band may be selected according to target tissue depth to perform depth calibration, while specific wavelengths within the second wavelength band exhibiting high absorption characteristics with respect to glucose are used for spectral identification. Concentration estimation is further performed based on multi-wavelength scattering signals. By integrating spectral information obtained from the first wavelength band and the second wavelength band, resolution and accuracy of glucose absorption signals can be significantly improved. In addition, by virtue of the wide-angle emission characteristic of light-emitting diodes, the present invention realizes a diffuse reflectance-based optical detection mechanism without requiring additional secondary optical focusing elements, thereby simplifying optical paths and structural design and enabling a miniaturized detection configuration. By combining the above-described optical design with advantages of high integration and low power consumption, the blood glucose monitoring module 100 proposed by the present invention can be effectively applied to the wearable device 1000, thereby providing a novel, high-performance, and portable solution for blood glucose concentration detection.

The above embodiments are provided to illustrate the implementations of the present invention and to explain its technical features, and are not intended to limit the scope of the present invention. Any modifications or equivalent arrangements that can be easily accomplished by those skilled in the art fall within the scope of the present invention, and the scope of the present invention should be defined by the claims.

What is claimed is:

1. A blood glucose monitoring module with a composite-wavelength light source, comprising:
a light-emitting component including a plurality of light source elements configured to emit a plurality of light beams toward a subject, wherein the light beams have wavelengths in a range from about 700 nanometers to about 1700 nanometers, and the light beams include at least one first wavelength band and at least one second wavelength band, wherein the at least one first wavelength band is configured to calibrate depth-dependent optical properties of skin tissue, and the at least one second wavelength band is configured to detect absorption intensity associated with blood glucose; and
a light-sensing component disposed adjacent to the light-emitting component and configured to receive a plurality of diffusely reflected optical signals reflected from the subject after being illuminated by the light beams, so as to acquire optical characteristics indicative of a blood glucose concentration of the subject.

2. The blood glucose monitoring module of claim 1, wherein the at least one first wavelength band has a wavelength range from about 700 nanometers to about 1100 nanometers.

3. The blood glucose monitoring module of claim 1, wherein the at least one second wavelength band has a wavelength range from about 1100 nanometers to about 1700 nanometers.

4. The blood glucose monitoring module of claim 1, further comprising a substrate, wherein the light-emitting component and the light-sensing component are disposed on the same side of the substrate.

5. The blood glucose monitoring module of claim 4, wherein the light-emitting component and the light-sensing component are disposed corresponding to each other, the light-emitting component is disposed at one end of the same side of the substrate, and the light-sensing component is disposed at the other end of the same side of the substrate.

6. The blood glucose monitoring module of claim 1, wherein the light source elements comprise a plurality of light-emitting diodes.

7. The blood glucose monitoring module of claim 1, wherein the light-sensing component comprises a plurality of photodiodes.

8. The blood glucose monitoring module of claim 1, further comprising an optical filter configured to selectively pass the at least one first wavelength band or the at least one second wavelength band.

9. The blood glucose monitoring module of claim 1, wherein the light-sensing component further comprises a data processing unit configured to receive and analyze spectral signals of the plurality of diffusely reflected optical signals, and to output data corresponding to the blood glucose concentration by comparing the spectral signals with reference spectra.

10. A wearable device, comprising:
a blood glucose monitoring module according to claim 1;
a housing configured to accommodate the blood glucose monitoring module;
a power element disposed within the housing, the power element being coupled to the blood glucose monitoring module and configured to supply power; and
at least one printed circuit board assembly disposed within the housing, the at least one printed circuit board assembly being coupled to the blood glucose monitoring module and configured to control operation of the blood glucose monitoring module.
